(19) United States
(12) Patent Application Publication
(10) Pub. No.: US 2025/0241724 A1
(43) Pub. Date: Jul. 31, 2025

(54) STORAGE SYSTEM

(71) Applicant: Daves Medical LLC, Port Orange, FL (US)

(72) Inventor: David A. Dufek, Daytona Beach, FL (US)

(21) Appl. No.: 19/008,254

(22) Filed: Jan. 2, 2025

Related U.S. Application Data
(60) Provisional application No. 63/625,093, filed on Jan. 25, 2024.

(51) Int. Cl. A61B 50/30; A61G 99/00

(52) U.S. Cl. CPC A61B 50/30; A61G 99/00

(57) ABSTRACT

A storage system for a patient support apparatus includes a plurality of couplers interconnected and arranged in a plane to form a plurality of connection locations and being bounded by an outer boundary sized to fit in and to be fully contained in a section of a patient support at a lower surface of the patient support when mounted to the patient support, which are for releasably attaching one or more storage modules at selected connection locations of the plurality of connection locations. The storage system further includes at least one storage module removably attached to a selected coupler or selected couplers of the plurality of couplers in selected locations of the plurality of connection locations to removable secure the at least one module to the upper frame of the patient support, with the storage module forming a compartment for storing one or more articles.

### CROSS REFERENCE TO RELATED APPLICATION

The present application claims the priority benefit of U.S. provisional patent application Ser. No. 63/625,093, filed on Jan. 25, 2024, which is incorporated by reference herein in its entirety.

### TECHNICAL FIELD AND BACKGROUND

The present disclosure relates to a storage system and, especially, to a storage system for use with a patient support apparatus, such as an emergency medical services (EMS) cot, a stretcher, chair, operating room table, or a hospital bed.

When handling patients, access to accessories can be critical. While numerous solutions have been developed, the solutions are often time consuming to use and also expensive. When dealing with critically ill patients, time is of the essence, and any second saved can make a difference.

### SUMMARY

Accordingly, a storage system for a patient support apparatus is disclosed that is simple but effective. The system includes a plurality of couplers interconnected and arranged in a plane to form a plurality of connection locations. The couplers are bounded by an outer perimeter (imaginary or tangible) sized to fit in and be fully contained in a section of the patient support apparatus beneath the patient support that supports the patient thereon. The plurality of couplers is configured to releasably attach a plurality of modules at selected connection locations of the plurality of connection locations. The system further includes at least one module releasably attached to a selected coupler or selected couplers of the plurality of couplers in selected locations of the plurality of connection locations to removable secure the at least one module to the patient support.

In one aspect, the patient support is formed by a tubular frame and a web that extends between the tubular frame to form an upper surface for supporting a patient thereon and a lower surface facing a base of the patient support apparatus. The plurality of couplers is arranged in a plane that extends between opposed sides of the tubular frame adjacent the web at the lower surface of the web.

In another embodiment, a storage system for a patient support apparatus where the patient support apparatus has an upper frame that forms a patient support and a base for supporting the patient support on a floor or the ground. The patient support has a tubular frame and a web that extends between the tubular frame. The web forms an upper surface for facing and supporting a patient thereon and a lower surface for facing the base, with the lower surface and the tubular frame forming a space there between. The storage system includes a panel sized to fit and configured to be secured and fully contained in the space and to extend between opposed sides of the tubular frame adjacent or abutting the lower surface of the web. The panel has a plurality of connection locations formed by a plurality of couplers arranged in a plane at the panel for releasaby coupling a plurality of modules to the panel, and the panel having an outer perimeter sized to substantially traverse the space between the opposed sides of the frame.

In a further aspect of the above, the storage system may include one or more modules that are releasably attached to a selected coupler or selected couplers of the plurality of couplers in selected locations of the plurality of connection locations to releasably secure the plurality of modules to the upper frame of the patient support.

In yet another embodiment, a storage system for a patient support apparatus, which includes an upper frame that forms a patient support and a base for supporting the patient support on a floor or the ground, with the upper frame having a tubular perimeter frame, a transverse frame member that extends between opposed sides of the tubular perimeter and a web that extends between opposed the opposed sides of the tubular frame and adjacent the transverse frame member. The web is formed by a plate that forms an upper surface for facing and supporting a patient thereon and a lower surface for facing the base. The lower surface, the transverse frame member, and the tubular perimeter frame together form a space there between. The storage system includes a panel that is sized to fit and is configured to be secured in the space and to extend between opposed sides of the frame and beneath the transverse member and to be adjacent or abutting the lower surface of the patient support. The panel includes a plurality of connection locations formed by a plurality of couplers arranged in a plane at the panel for releasaby coupling a plurality of modules to the panel, and with the panel having an outer perimeter sized to substantially traverse the space between the opposed sides of the tubular frame.

In one aspect of the above, the storage system also includes a plurality of modules removably attached to selected couplers of the plurality of couplers in selected locations of the plurality of connection locations to releasably secure the plurality of modules to the upper frame of the patient support.

In any of the above panels, the plurality of couplers are formed by the panel.

In any of the above panels, the panel may comprise a core layer and a hook and loop fastener layer secured to the core layer to form the plurality of couplers. For example, the hook and loop fastener layer comprises at least two hook and loop fastener strips or patches.

In one aspect, the hook and loop fastener layer may be glued, stitched, or welded or otherwise secured to the core layer.

In one aspect, the core layer comprises a plastic layer, such a corrugated plastic layer.

In yet a further aspect, the hook and loop fastener layer comprises a first hook and loop fastener layer, and the panel includes a second the hook and loop fastener layer, with the core layer sandwiched between the first and second hook and loop fastener layers so that the panel is reversible or so that the panel can be secured to the patient support apparatus using the second hook and loop fastener layers.

In any of the above modules, the modules may comprise storage modules.

In any of the above, the panel includes a plurality of connectors to secure the panel to the patient support. Suitable connectors may include straps, including straps that are secured to the panel.

### DETAILED DESCRIPTION OF THE FIGURES

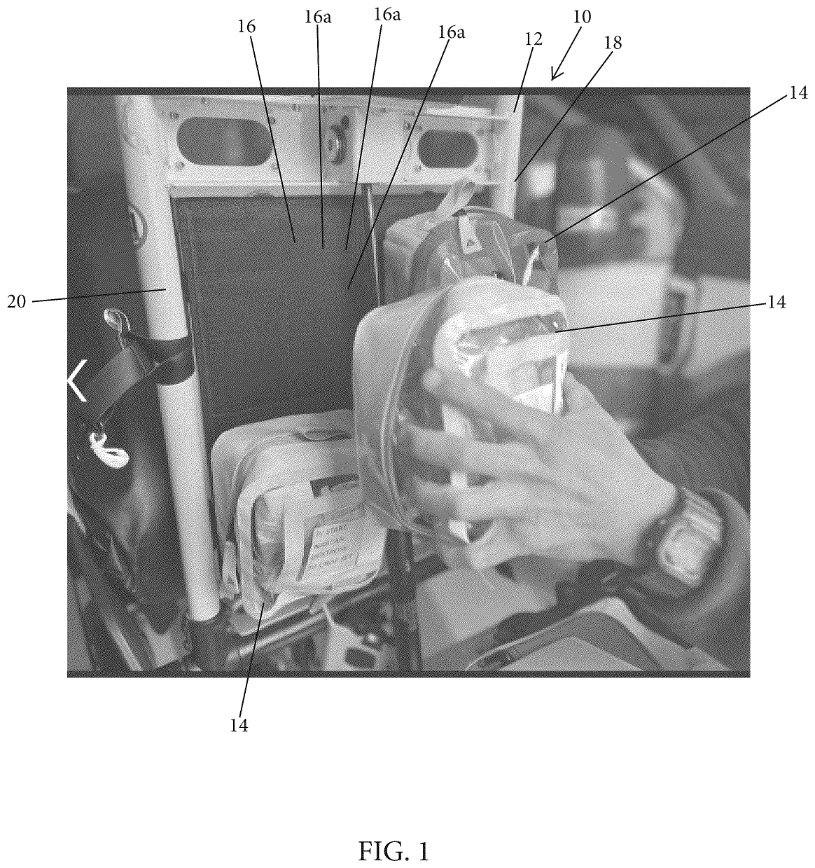

FIG. 1 is a perspective view of a storage system of the present disclosure shown mounted to a patient support apparatus;

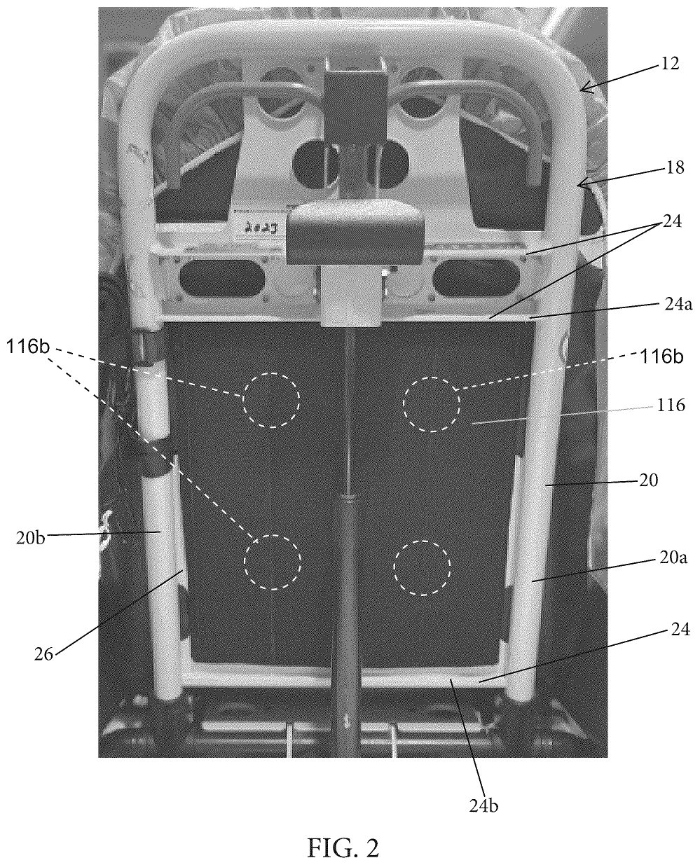

FIG. 2 is a similar view to FIG. 1 with the modules removed for clarity illustrating an alternate embodiment of a panel;

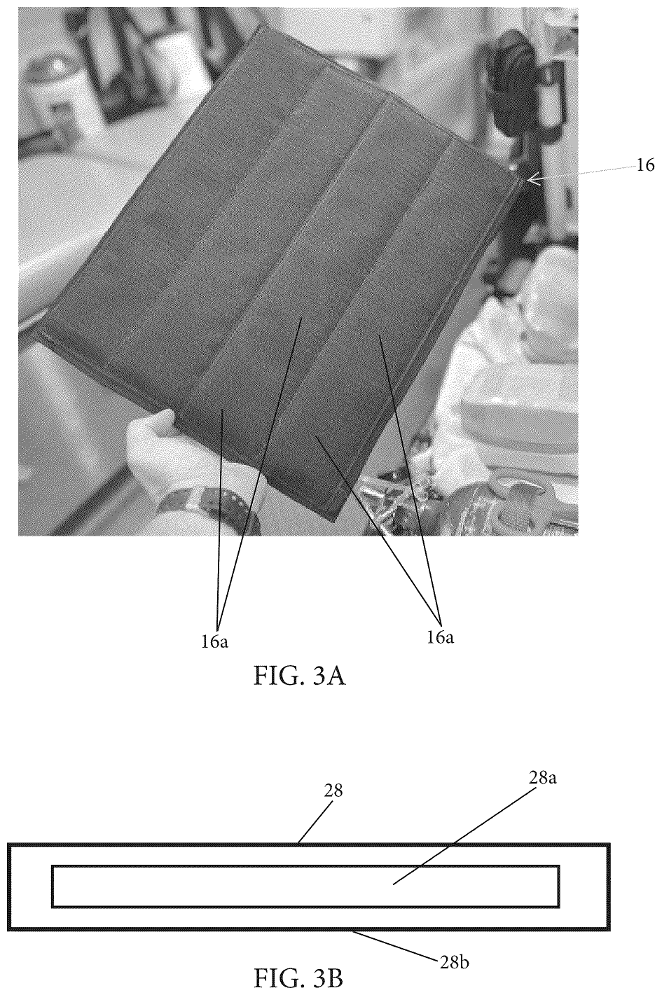

FIG. 3A is a perspective view of a panel of the storage system of FIG. 1;

FIG. 3B is a cross-section of the panel of FIG. 3A;

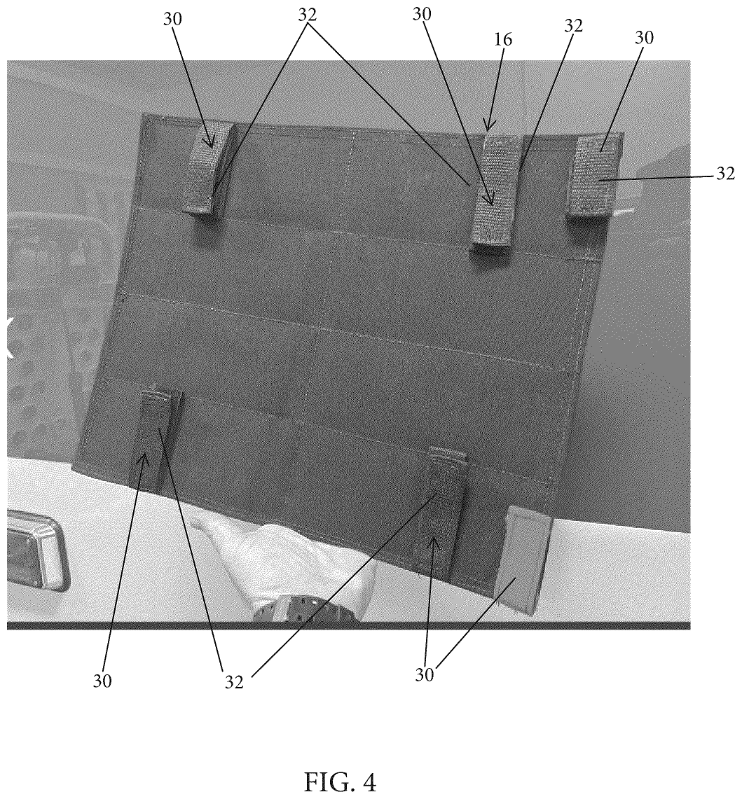

FIG. 4 is a back perspective view of the panel of FIG. 3A;

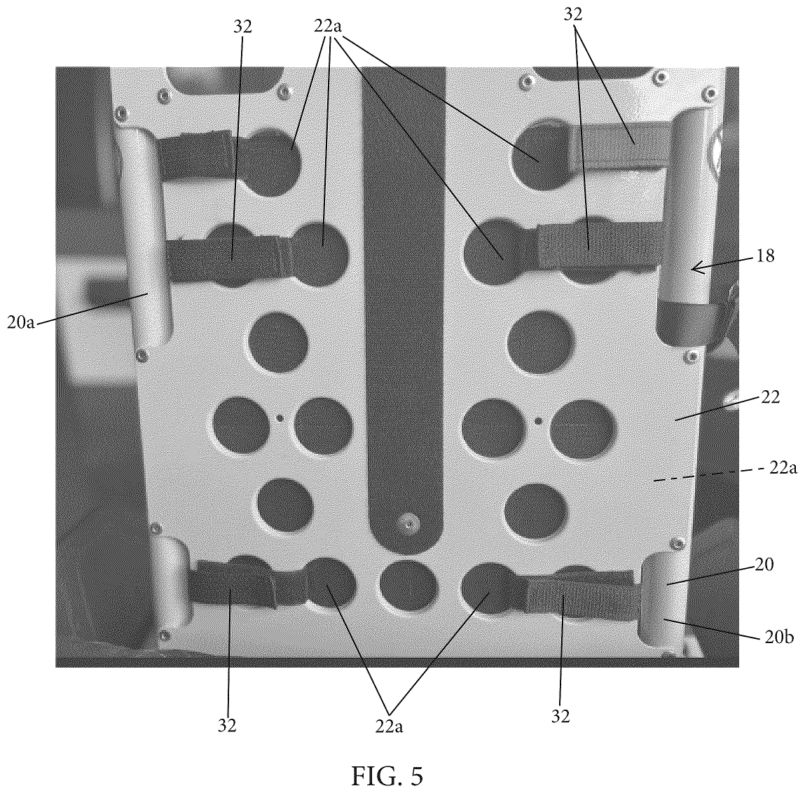

FIG. 5 is a back view of the Fowler section of the patient support apparatus of FIG. 1 illustrating how the panel may be secured to the Fowler section;

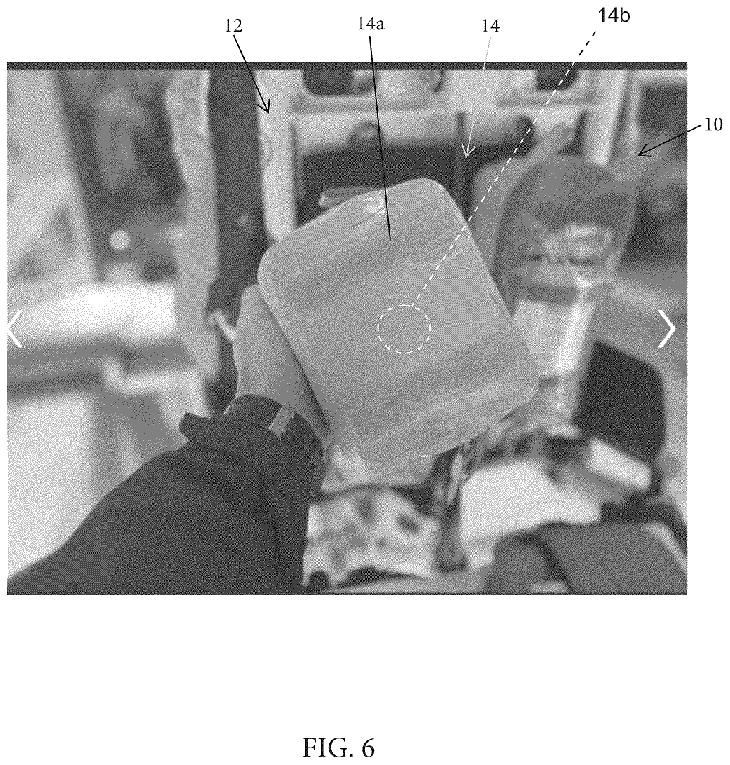

FIG. 6 is a rear perspective view of one of the modules that may be incorporated into the storage system;

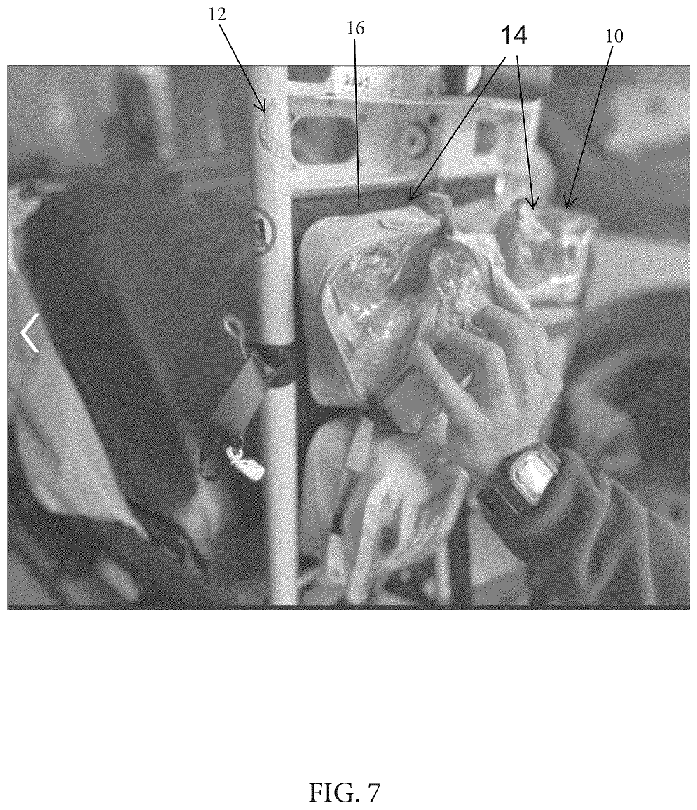

FIG. 7 is a perspective view illustrating the use of the storage system;

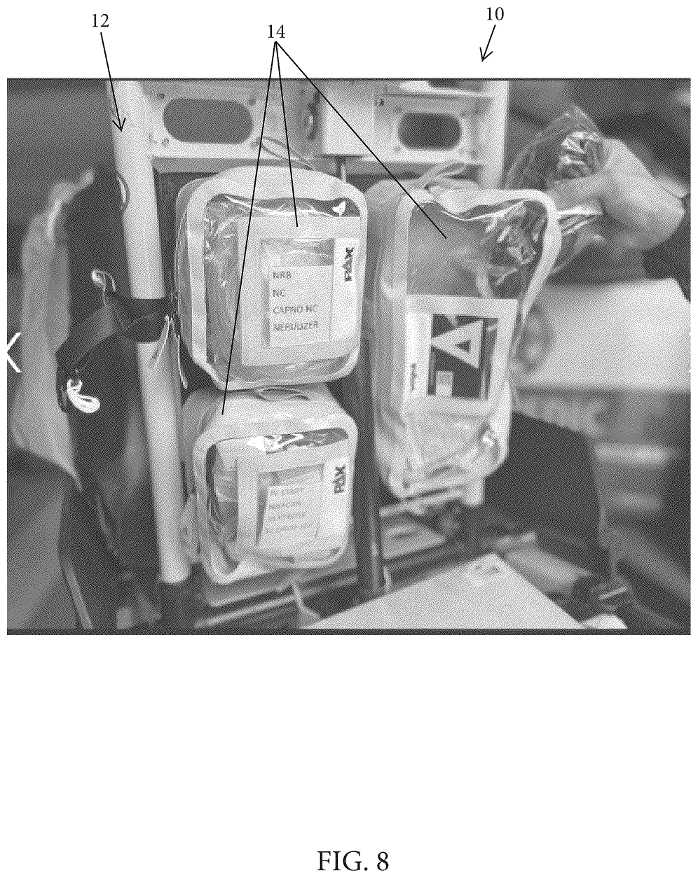

FIG. 8 is a perspective view illustrating the use of the storage system with another

module; and

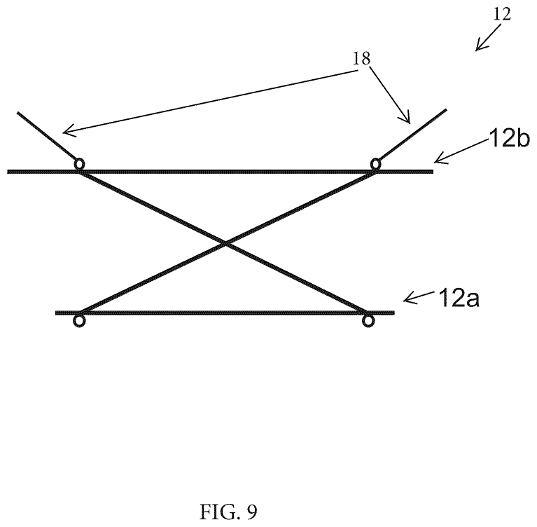

FIG. 9 is a side elevation view of an exemplary embodiment of a patient support apparatus that is suitable for use with the storage system.

### DETAILED DESCRIPTION OF THE PREFERRED EMBODIMENTS

Referring to FIG. 1, the numeral 10 generally designates a storage system, which is particularly suitable for use with a patient support apparatus 12. The patient support apparatus 12 may comprise an EMS cot, a stretcher, a hospital bed, a patient transport device, such as a transport chair, a chair, an operating room table, or other patient support apparatuses. As will be more fully described below, storage system 10 allows a caregiver to quickly secure or retrieve modules 14 from the patient support apparatus 12 so that a caregiver can customize the modules that they wish to have accessible at the patient support apparatus. Further, as will be more fully described below, the modules may be secured by simply placing the module in contact with one or more couplers that are located at the patient support apparatus or retrieved by simply pulling on the module.

As best seen in FIGS. 1 and 2, storage system 10 includes a panel 16. Panel 16 is a planar panel that includes a plurality of couplers 16a that are arranged in the panel to form a plurality of connection locations and further are arranged in a plane. The couplers 16a are, therefore, bounded by the outer perimeter of the panel. As will be more fully described below, panel 16 is releasably mounted to patient support apparatus 12 and recessed within a section of the patient support apparatus 12. In this manner, when the panel 16 is mounted to the patient support apparatus 12, the couplers may also be recessed in the patient support apparatus 12 and, further, located for easy access by a caregiver who can then easily secure one or more modules to the panel or retrieve one or more modules from the panel and, hence, from patient support apparatus using a single hand.

As best seen in FIGS. 1, 2, and 9, patient support apparatus 12 is configured as an EMS cot includes a base 12a and patient support 12b that includes one or more articulatable patient support sections 18. As noted above, the patient support apparatus may be configured in other forms of patient support apparatuses. In the illustrated embodiment, articulatable patient support section 18 is formed from a tubular frame 20 and a web 22 (FIG. 5) that extends between the opposite sides 20a and 20b of tubular frame 20 to form an upper surface 22a to support a patient thereon. Further, in the illustrated embodiment, the tubular frame 20 and web 22 may be in the form what is often referred to as a “Fowler section” of the patient support apparatus 12 in that it supports the back of the patient. Although not described herein in detail, articulatable patient support section 18 may form a leg section, which together with a seat section and the Fowler section provide full support for the patient, and which may be similarly formed from a tubular frame and web. For the purposes of this description, the illustration of the storage system will be made in reference to the Fowler section; however, it should be understood that the leg section or other sections of the patient support apparatus may also be used to mount the storage system.

Referring into FIG. 2, tubular frame 20 may be reinforced by transverse members 24 that extend between the opposed sides 20a, 20b of the tubular frame 20 beneath web 22, which together with opposed sides 20a and 20b of tubular frame member 20 and web 22 form a space 26 in which panel 16 may be located and recessed, and optionally fully recessed. Further, panel 16 maybe sized so that it extends across space 26 between opposed sides 20a, 20b of tubular frame 20 and, further, between upper transverse frame member 24a and lower transverse frame member 24b, leaving only small gaps (e.g., ⅛-½ inch) therebetween, or may be sized to leave no gaps. Additionally, panel 16 may be located immediately adjacent web 22 so that it makes contact with web 22 on its inwardly facing side (side facing web 22). In addition, panel 16 is configured to be secured to web 22, as will be more fully described below.

To form the plurality of couplers, panel 16 may be formed using strips of hook and loop fasteners 28, such as strips or patches of VELCRO, including side-by-side strips of hook and loop fasteners, which are joined together, for example, by stitching, welding or gluing. Alternately, or in addition, snaps or magnets may be used instead or in a combination of fasteners.

Optionally, as best seen in FIG. 3A, the entire outwardly facing side of panel 16 may include hook and loop fasteners, such that panel 16 includes a plurality of closely spaced couplers across its full width and height to form a plurality of attachment locations across its entire outwardly facing side to couple to modules 14. In this manner, panel 16 includes a plurality of closely spaced couplers across its full width and height to form a plurality of attachment locations across its entire outwardly facing side.

Alternately, a panel 116 may have an outwardly facing side formed from a smooth material, such as a plastic sheet, such as nylon or PVC or the like, with magnets or snaps 116b mounted to the panel (FIG. 2). For example, magnets may be mounted beneath the outwardly facing surface of the panel (including by molding), which couple to magnetic or magnetized inserts provided in the modules 14. In this manner, panel 116 includes a plurality of discrete, but spaced couplers across its width and height to form a plurality of attachment locations across its outwardly facing side. It should be understood that the magnets may be instead mounted to the modules, and the magnetic or magnetized inserts may be mounted to the panel.

Referring to FIG. 3B, to provide stiffness to the panel 16 or 116, panel 16 or 116 may include a core layer 28a, such as a layer formed from plastic, such as a corrugated plastic layer. The hook and loop fasteners 28 may be secured to the core layer 28a using stitching, gluing, welding, or the like. Similarly, the snaps or magnets may be secured on or in the core layer 28a via fasteners or glue or welding. Further, the panel 16 or 116 may include an intermediate layer between the fastener layer (hook and loop, snaps or magnets) and the core layer, such as a non-liquid permeable fabric layer, including a non-woven fabric or a rubberized fabric, to prevent fluids from flowing into the modules when secured to the panel.

Optionally, the inwardly facing side (side that faces web 22) of panel 16 or 116 may also include a layer of hook and loop fasteners (or other couplers as described above) and an optional intermediate layer to prevent fluid from flowing through or into the core layer. For example, this may allow the panel to be reversible or to be used to secure the panel to the web, for example, by fasteners secured to the lower surface of web 22. In one embodiment, the magnets may be configured to secure the panel to the web 22. For example, magnetic or magnetized inserts may be provided in web 22, for example, in selected openings or recesses 22a in the web 22, more fully described below. Alternately or in addition, the panel 16 may be secured to web 22 by connectors 30, such as straps 32, which are described more fully below.

In one embodiment, and referring to FIG. 4, as noted, panel 16 may include a plurality of connectors 30 for connecting panel 16 or 116 to the web 22 of patient support apparatus 12. For example, connectors 30 may comprise straps 32 that are secured on one end to panel 16 or 116 either by stitching, gluing, or welding, or via hook and loop fastener patches or strips that are secured to the respective straps so that they have adjustable attachment points to accommodate different patient support apparatuses. Additionally, the free or distal ends of the respective straps may include hook and loop fastener strips or patches to allow them to be looped around the tubular frame 20 and/or through the web 22 to secure the panel in place, as shown in FIG. 5. Alternately, or in addition, hook and loop fastener patches or strips may be mounted to the web 22, as noted, to directly engage the inwardly facing side of panel 16, 116 and thereby secure panel 16 or 116 to web 22.

Referring again to FIG. 5, as noted, web 22 may include openings 22a, which allow the straps 32 to pass through openings 22a formed in web 22 and extend over the edge of the web 22 (or around the tubular frame) to thereby secure the panel 16 in place. Straps 32 may have snaps for engaging snaps applied to the patient support apparatus to secure panel 16 or 116 in place or, as noted, hook and loop patches or strips.

As noted above, storage system 10 allows one or more modules 14 to be releasably secured to panel 16 or 116 to allow a user to store the one or more modules 14 at the patient support, for example, beneath the patient support surface formed by upper surface 22a of web 22. Each module 14 may have a hook and loop fastener strip 14a (FIG. 6) at its base side for facing and securing the module to the panel 16. Additionally, with the construction described above, the modules may be secured to panel 16 by simply contacting the panel with the module (and possibly by applying light pressure to the module) and, therefore, can be done using a single hand. Alternately (or in addition), each module 14 may have one or more magnetic or magnetized inserts or snaps 4b at its base side for facing and securing the module to the panel 116. Similarly, in this manner, the modules may be secured to panel 116 by simply contacting the panel with the module (and possibly by applying light pressure to the module) and, therefore, can be done using a single hand. It should be understood that magnets or snaps may also be included in panel 16 such that panel 16 has multiple types of couplers.

Further, the modules 14 may comprise storage modules for storing articles, such as EMS accessories, including bandages, wipes, gloves, masks, restraints, batteries, medical devices, including syringes, medical testing devices, and medicines, each with a compartment and a flap that closes the compartment using a zipper or the like. The compartments may be formed from transparent panels so that a user can observe when a supply of the accessories stored in a respective compartment is running low and/or so they can see what accessory is stored therein. When secured to the panel, a user can then access the inside of module via the module's zipper, such as shown in FIGS. 7 and 8.

As noted above the type of patient support apparatus may vary. In the illustrated embodiment, as noted, the patient support apparatus is configured as an EMS cot. Referring to FIG. 9, the patient support apparatus includes tubular frames and webs, which are joined together to form the sections 18 of patent support apparatus 12. Sections 18 are pivotal so when that section is raised, a user will have easy access to the underside or back of the panel for securing or retrieving modules therefrom. Further, the modules may be sized so that when the frame section is lowered, the modules will not be crushed or will not form an interference for the patient support apparatus.

While a solid panel with a continuous physical outer perimeter is illustrated, the panel may be formed be interconnected strips or patches, with an outer boundary (at least part physical and part imaginary (the space between the strips or patches)) that is still sized to fit in space 26. Optionally, in addition, while the shape of the panel is illustrated as rectangular, it may have an irregular shape to accommodate other components that may be mounted beneath the respective section of the patient support. In an irregularly shaped panel, portions of the panel may be removable or missing due to accommodate an interference, with the outer boundary being formed from a physical outer perimeter and an imaginary outer perimeter that is a projection of where the panel would extend but for the portion or portions of the panel that are missing.

It should be understood the number, shape, and size of the modules may be varied. In some instances, one module may extend across the majority of the width or height of the panel. In other instances, the modules may be sized so that two of more modules may be mounted to the panel in a side-by-side relationship to span (together) the width and/or the height of the panel.

1. A storage system for a patient support apparatus, the patient support apparatus having an upper frame that forms a patient support and a base, the patient support having an upper surface for facing and supporting a patient thereon and a lower surface for facing the base, the patient support including a section, said storage system comprising:
a plurality of couplers interconnected and arranged in a plane to form a plurality of connection locations and being bounded by an outer boundary sized to fit in and to be fully contained in the section of the patient support at the lower surface of the patient support when mounted to the patient support, and said plurality of couplers for releasably attaching one or more modules at selected connection locations of said plurality of connection locations; and
at least one storage module removably attached to a selected coupler or selected couplers of said plurality of couplers in selected locations of said plurality of connection locations to removable secure said at least one module to the upper frame of the patient support, and the storage module forming a compartment for storing one or more articles.

2. The storage system according to claim 1, further comprising a plurality of the storage modules removably attached to selected couplers of said plurality of couplers in selected locations of said plurality of connection locations to releasably secure said plurality of storage modules to the upper frame of the patient support.

3. The storage system according to claim 2, further comprising a panel, and wherein said plurality of couplers are formed at said panel.

4. The storage system according to any one of claim 3, wherein said panel includes a plurality of connectors to secure the panel to the patient support.

5. The storage system according to claim 4, wherein said plurality of connectors comprises straps.

6. The storage system according to claim 2, wherein said panel comprises a core layer and said couplers secured to said core layer to form said plurality of connection locations.

7. The storage system according to claim 6, wherein said panel includes one or more hook and loop fastener layers glued, stitched, or welded to said core layer.

8. The storage system according to claim 7, wherein said core layer comprises a plastic layer, such a corrugated plastic layer.

9. The storage system according to claim 7, wherein said hook and loop fastener layer comprises a first hook and loop fastener layer, further comprising a second said hook and loop fastener layer, and said core layer sandwiched between said first and second hook and loop fastener layers.

10. The storage system according to claim 2, wherein said couplers comprise couplers selected from a group consisting of magnets, snaps, and hook and loop fastener patches or strips.

11. A storage system for a patient support apparatus, the patient support apparatus having an upper frame that forms a patient support and a base for supporting the patient support on a floor or the ground, the patient support having a tubular frame and a web that extends between the tubular frame, the web forming an upper surface for facing and supporting a patient thereon and a lower surface for facing the base, and the lower surface and the tubular frame forming a space there between, said storage system comprising:
a panel sized to fit and configured to be secured and fully contained in the space and to extend between opposed sides of the tubular frame adjacent or abutting the lower surface of the web when mounted to the patient support; and
said panel having a plurality of connection locations formed by a plurality of couplers arranged in a plane at said panel for releasaby coupling a plurality of storage modules to said panel, and said panel having an outer boundary sized to substantially traverse the space between the opposed sides of the frame when mounted to the patient support.

12. The storage system according to claim 11, further comprising a plurality of the storage modules removably attached to selected couplers of said plurality of couplers in selected locations of said plurality of connection locations to releasably secure said plurality of storage modules to the upper frame of the patient support.

13. The storage system according to claim 12, wherein said couplers comprise couplers selected from a group consisting of magnets, snaps, and hook and loop fastener patches or strips.

14. The storage system according to claim 13, wherein said panel comprises a core layer, and said couplers being secured to said core layer to form said plurality of connection locations.

15. The storage system according to claim 14, wherein said core layer comprises a plastic layer.

16. The storage system according to claim 15, wherein said plastic layer comprises a corrugated plastic layer.

17. A storage system comprising:
a patient support apparatus, said patient support apparatus having an upper frame and a base, said base for supporting said patient support on a floor or the ground, said upper frame having a tubular perimeter frame, a transverse frame member extending between opposed sides of said tubular perimeter and a web extending between opposed sides of said tubular frame and adjacent said transverse frame member, said web being formed by a plate, said plate forming an upper surface for facing and supporting a patient thereon and a lower surface for facing said base, and said lower surface, said transverse frame member, and said tubular perimeter frame forming a space there between;
a panel, said panel being sized to fit and being secured in said space, said panel extending between said opposed sides of said frame and beneath said transverse member and being adjacent or abutting said lower surface of said patient support; and
said panel having a plurality of connection locations formed by a plurality of couplers arranged in a plane at the panel for releasaby coupling a plurality of storage modules to said panel, and said panel having an outer perimeter sized to substantially traverse said space between said opposed sides of said tubular frame; and
a plurality of the storage modules removably attached to selected couplers of said plurality of couplers in selected locations of said plurality of connection locations to releasably secure said plurality of storage modules to the upper frame of the patient support.

18. The storage system according to claim 17, wherein said couplers comprise couplers selected from a group consisting of magnets, snaps, and hook and loop fastener patches or strips.

19. The storage system according to claim 18, wherein said panel comprises a core layer, and said couplers being secured to said core layer to form said plurality of connection locations.

20. The storage system according to claim 19, wherein said core layer comprises a plastic layer.
